(19) United States
(12) Patent Application Publication
(10) Pub. No.: US 2006/0111452 A1
(43) Pub. Date: May 25, 2006

(54) Process for making silicone emulsions

(76) Inventors: Frank William Wallace, Brussels (BE); Henri Schirosi, Vitrival (BE)

Correspondence Address: DOW CORNING CORPORATION CO1232, 2200 W. SALZBURG ROAD, P.O. BOX 994, MIDLAND, MI, 48686-0994, US

(21) Appl. No.: 10/537,573

(22) PCT Filed: Feb. 4, 2004
(86) PCT No.: PCT/EP04/01738

§ 371 Date: Jun. 3, 2005

(30) Foreign Application Priority Data
Feb. 7, 2003 (GB) 0302840.4

(51) Int. Cl. B01F 3/08; C08L 83/04; A61K 8/00

(52) U.S. Cl. 516/53; 524/863

(57) ABSTRACT

An emulsion of an organo-functional polysiloxane is prepared by emulsifying a silanol-functional polysiloxane (I) in water, adding an organofunctional silane of the formula X-A-Si(R)ₙ(OR′)₃₋ₙ(II), where X represents an organic functional group; A represents a divalent organic linkage; each R represents a hydrocarbyl or substituted hydrocarbyl radical; each R′ represents hydrogen or an aklyl or acyl group; and n=′, 1 or 2, to the aqueous phase of the resulting emulsion and reacting the —OR′ groups of (II) with the silanol groups of the polysiloxane (I) to form the organo-functional polysiloxane.

### FIELD OF THE INVENTION

This invention relates to the production of silicone oil in water emulsions, useful for example in toiletry and cosmetic products such as shampoos, conditioners and skin creams, textile process additives such as hydrophilic/hydrophobic modifiers and softeners, and automotive care and household cleaning products. In particular it relates to the production of emulsions of organo-functional polysiloxanes, that is polysiloxanes containing functional organic groups such as amine, amide, epoxide, alcohol or thiol groups.

### BACKGROUND TO THE INVENTION

U.S. Pat. No. 6,239,211 describes the production of emulsions of amino-functional polysiloxanes by emulsifying low molecular weight or cyclic silicones and then reacting with amino-silanes at high temperature. U.S. Pat. No. 6,090,885 describes incorporation of amine functionality in linear polyorganosiloxane in the presence of cationic surfactant. U.S. Pat. No. 4,600,436 describes an aminofunctional silicone emulsion prepared from water, emulsifier, diorganopolysiloxane fluid, aminofunctional silane, and optionally a polymerisation catalyst, by emulsion polymerisation, and teaches that the emulsion polymerized polysiloxane emulsion can be stripped of cyclic or other low molecular weight siloxanes from which it was prepared.

U.S. Pat. No. 6,090,885 describes a process in which a hydroxy-stopped polydimethylsiloxane is emulsified in water with cyclic polyorganosiloxanes and polymerized to form a hydroxy end-stopped polydimethylsiloxane emulsion before being reacted with an aminofunctional silane. U.S. Pat. No. 6,552,122 also describes a process of reacting an aminofunctional silane with a polydimethylsiloxane emulsion that has been preformed by emulsion polymerisation of cyclic polyorganosiloxanes.

For use in the personal care industry, it is preferred that emulsions contain reduced quantities of the cyclic siloxane octamethylcyclotetrasiloxane (D4) due to its classification as a reproductive hazard. It is an object of the present invention to prepare emulsions of organo-functional polysiloxanes, especially amino-functional polysiloxanes, containing a lower level of D4 than the emulsions of amino-functional polysiloxanes prepared by emulsion polymerisation as described above, without needing stripping.

### SUMMARY OF THE INVENTION

A process according to the invention for the preparation of an emulsion of an organo-functional polysiloxane comprises mechanically emulsifying a silanol-functional polysiloxane (I) in water in the absence of any basic or acidic catalyst for silanol polycondensation, adding an organofunctional silane of the formula X-A-Si(R)ₙ(OR′)₃₋ₙ (II), where X represents an organic functional group; A represents a divalent organic linkage; each R represents a hydrocarbyl or substituted hydrocarbyl radical; each R′ represents hydrogen or an alkyl or acyl group; and n=0, 1 or 2, to the aqueous phase of the resulting emulsion and reacting the —OR′ groups of (II) with the silanol groups of the polysiloxane (I) to form the organo-functional polysiloxane.

### DETAILED DESCRIPTION OF THE INVENTION

The silanol-functional polysiloxane (I) is preferably a substantially linear polydiorganosiloxane fluid such as polydimethylsiloxane, although branched polysiloxanes can also be used. The silanol groups are preferably terminal groups on the polysiloxane chain. The polysiloxane fluid can for example have a viscosity of at least 0.02 Pa·s up to 1000 Pa·s (20 up to 1000000 cps), preferably 0.5 to 40 Pa·s. Most preferably the silanol-functional polysiloxane (I) has a molecular weight that is near the desired final molecular weight of the desired organofunctional polysiloxane. Both the emulsification of (I) and the reaction with the organofunctional silane (II) are preferably carried out under conditions which do not promote fast polycondensation of the polysiloxane (I).

The silanol-functional polysiloxane (I) is mechanically emulsified in water in the absence of any basic or acidic catalyst for silanol polycondensation. The silanol-functional polysiloxane (I) is preferably emulsified continuously, although it can alternatively be emulsified batchwise. In one preferred procedure the silanol-functional polysiloxane (I), at least one surfactant and water are continuously fed to a high shear mixer in such proportions as to form a viscous oil in water emulsion which is continuously withdrawn from the mixer and is diluted before addition of the organofunctional silane (II).

The amount of surfactant is generally at least 0.2% by weight based on the silanol-functional polysiloxane (I), preferably at least 0.5%, for example from 2% up to 10 or 20%. The amount of water present, including any water present in the surfactant composition, is generally at least 0.5% based on the polysiloxane fluid, preferably at least 1% up to 10 or 20% or even 30%. The polysiloxane content of the mixture fed into the high shear mixer is preferably from 70 to 99% by weight, most preferably 80 to 98%. At these proportions the polysiloxane, surfactant and water form a non-Newtonian “thick phase” emulsion, which has a very high viscosity at low shear rates, but mixtures with this high polysiloxane content emulsify more readily to small particle size than a more dilute mixture.

Mechanical emulsion via such a “thick phase” is most effectively carried out as a continuous process. A particularly preferred procedure is described in WO-A-02/42360. The high shear mixer can for example be an in-line, dynamic rotor/stator device such as those sold under the Trade Marks “TK Products Homomic Line Mill” or “Bematek” or “Greerco” or “Ross”, often referred to as a colloid mill, or a rotary disc mixer of the type described in JP-A-2000-449, or a twin screw compounder of the type used for plastics extrusion.

The surfactant used for emulsification of the silanol-functional polysiloxane (I) is preferably one or more non-ionic surfactant. Examples of non-ionic surfactants include polyoxyalkylene alkyl ethers such as polyethylene glycol long chain (9-22C, especially 12-14C) alkyl ether, polyoxyalkylene sorbitan ethers, polyoxyalkylene alkoxylate esters, polyoxyalkylene alkylphenol ethers, ethylene oxide propylene oxide copolymers, polyvinyl alcohol, glyceride esters and alkylpolysaccharides. Non-ionic surfactants are generally unlikely to catalyse polycondensation of the polysiloxane.

Ionic surfactants such as cationic, amphoteric and/or anionic surfactants can alternatively be used. Examples of cationic surfactants include quaternary ammonium salts such as 8-22C alkyl trimethyl ammonium halides, 8-22C alkyl dimethyl benzyl ammonium halides or di(8-22C alkyl) dimethyl ammonium halides. Examples of suitable amphoteric surfactants include cocamidopropyl betaine, cocamidopropyl hydroxysulphate, cocobetaine, sodium cocoamidoacetate, cocodimethyl betaine, N-coco-3-aminobutyic acid and imidazolinium carboxyl compounds. Examples of anionic surfactants include alkyl sulfates such as lauryl sulfate, polymers such as acrylates/C₁₀₋₃₀ alkyl acrylate crosspolymer, (6-20C alkyl) benzenesulfonic acids and salts, the sulfate esters of monoalkyl polyoxyethylene ethers, sulphonated glyceryl esters of fatty acids, and salts of sulphonated monovalent alcohol esters. Some anionic surfactants such as sulphonic acids have catalytic activity for condensation polymerisation of silanol-functional polydiorganosiloxanes. The catalytic activity can be suppressed by a neutralising agent such as an organic amine, for example triethanolamine, or an inorganic base such as sodium hydroxide. In general we prefer to avoid use of anionic surfactant unless it is desired to emulsify the silanol-functional polysiloxane (I) in a process including controlled polymerisation of (I).

If the silanol-functional polysiloxane (I) is emulsified as a “thick phase”, it is preferably diluted before addition of the organofunctional silane (II). The emulsion preferably has a concentration of 20-75% by weight polysiloxane (I) at the time it is reacted with the organofunctional silane (II). The “thick phase” can be diluted with water alone, or with a mixture of water and surfactant. The surfactant used in dilution can be of any of the types described above. The surfactant may be chosen to be the most compatible with the organofunctional silane (II). For example a cationic surfactant can be used in the dilution step when the organic functional group of (II) is an amino group. Alternatively, non-ionic surfactants are generally suitable for dilution.

The organofunctional silane of the formula X-A-Si(R)ₙ(OR′)₃₋ₙ (II) is most preferably an aminosilane. The invention is particularly suitable for the production of emulsions of amino-functional polysiloxane useful for example in toiletry and cosmetic products such as shampoos and skin creams. The organic functional group X is thus preferably a primary, secondary or tertiary amine group, for example —NH₂ or —NHC₂H₅, or can be a group including both primary and secondary amino such as —NHC₂H₄NH₂.

The organic functional group X can alternatively be an amide, epoxide, alcohol or thiol group.

The groups OR′ in (II) are preferably alkoxy groups, that is R′ is preferably an alkyl group, more preferably 1-4C alkyl. The group R, if present, is also preferably 1-4C alkyl. Most preferably each group R′ of silane (II) is a methyl radical. We have found that methoxy silanes are more reactive than ethoxy or higher alkoxy silanes, and aminosilanes containing methoxy groups are thus easier to incorporate into the polysiloxane. Particularly preferred examples of organofunctional silanes (II) include 3-aminopropyl trimethoxy silane and 3-(2-aminoethylamino)propyl trimethoxy silane.

The molar ratio of silanol groups of (I) to Si-bonded alkoxy or other groups OR′ of organosilane (II) is preferably in the range (0.4-1.5:1). In many cases it is preferred that the molar ratio of silanol groups of (1) to Si-bonded alkoxy groups of aminosilane (II) is less than 1:1, so that the main reaction taking place is capping of the silanol-functional polysiloxane (I) by the amino- or other organo-functional alkoxysilane (I). Molar ratios of silanol groups of (I) to Si-bonded alkoxy groups of aminosilane (II) greater than 1:1 may be preferred if it is desired that chain extension polymerisation of (I) should also take place to produce an organo-functional polysiloxane of greater degree of polymerisation than the starting silanol-functional polysiloxane (I).

When the organofunctional silane (II) is an amino- or amido-functional silane, the reaction with polysiloxane (I) is preferably carried out in the presence of a cationic surfactant is added to the emulsion no later than the addition of the organofunctional silane (II). When the polysiloxane (I) is emulsified as a “thick phase” and is diluted before reaction with the organofunctional silane (II), the cationic surfactant can be present in the water in the dilution step as described above. The cationic surfactant can alternatively be added with the aminosilane (II), or can be added to the dilute emulsion before addition of (II). The amount of cationic surfactant added can for example be 1 to 10% based on the total weight of siloxane reagents.

A base is preferably added to the emulsion to catalyse the reaction of the —OR′ groups of (II) with the silanol groups of the polysiloxane (I). The base can be added to the emulsion before, together with or after the organofunctional silane (II). The base is preferably an inorganic base such as sodium hydroxide or potassium hydroxide, or alternatively can be an amine such as triethanolamine. The amount of base is preferably that required to obtain pH of 9-13, most preferably 11-12.

The organofunctional silane (II) and the silanol-functional polysiloxane (I) are preferably reacted at a temperature below 40° C., most preferably below 30° C., for example at ambient temperature of 10-25° C. We have found that a low temperature of reaction between (I) and (II) is particularly effective in producing an emulsion having a low D4 level. The time of reaction can for example be 0.5 to 24 hours.

The emulsion of organo-functional polysiloxane produced by the process of the present invention generally contains less than 2% by weight cyclic polysiloxane, in particular less than 2% D4, based on the total weight of polysiloxane in the emulsion. When the temperature of reaction of (I) and (II) is kept below 30° C., emulsions of aminosiloxane containing less than 1% D4 can be produced.

The emulsions of the present invention have particular advantage for personal care applications, for example in toiletry and cosmetic products such as shampoos and skin creams, where there is a particular demand for emulsions of low D4 content, but are also advantageous for use in textile treatment, for example as fabric softeners, and for automotive care.

The invention is illustrated by the following Example, in which parts and percentages are by weight.

### EXAMPLE 1

60 parts of a substantially linear hydroxy-endblocked polydimethylsiloxane of viscosity 4000 mPa·s. was emulsified with 2.5 parts Renex 30 (Trade Mark) nonionic surfactant and 1.33 parts water via a continuous process using a high shear mixer as described with reference to FIG. 1 of WO-A-02/42360. The resulting thick phase emulsion was diluted with water batchwise to 50% silicone in a stirred reactor, and 5.87 parts Arquad 16-29 (Trade Mark) 30% active cationic surfactant was added. 0.5 parts 50% aqueous sodium hydroxide and 7 parts 3-(2-aminoethylamino)propyl trimethoxy silane were added. The emulsion was reacted for 6 hours at room temperature (23° C.). An aminosiloxane emulsion of particle size 200 nm was obtained. The D4 content of the final emulsion comprised 0.7% of the silicone phase.

Application testing on hair has shown equivalent performance to a commercial aminosiloxane emulsion of D4 content 6.6%.

### EXAMPLE 2

35 parts of a substantially linear hydroxy-endblocked polydimethylsiloxane of viscosity 4000 mPa·s. was emulsified with 2.9 parts Renex 30 (Trade Mark) nonionic surfactant, 1.7 parts Servamine KAC 458 (Trade Mark) Cationic Surfactant and 1.33 parts water via a batch process, consisting of a Klaussen (Trade mark) 10 Liter change-can mixer with a scraper blade and two high speed disperser discs. The mixture was diluted with 55.8 parts water. 1.8 parts Arquad 16-29 (Trade Mark) 30% active cationic surfactant, 0.5 parts 3-(2-aminoethylamino)propyl trimethoxy silane and 0.5 parts NaOH 40% solution were added with mixing. The emulsion was reacted at 8 hours at room temperature (23° C.), then neutralized with 0.5 parts glacial acetic acid. An aminosiloxane emulsion with median particle size of 170 nm was obtained. The amine content by aminopotentiometric titration was 0.124 meq/g; the final pH was 7.6, and the viscosity of the extracted polymer phase was 6,500 cp; the D4 content was 0.76 wt % of the silicone phase.

1. A process for the preparation of an emulsion of an organo-functional polysiloxane comprising mechanically emulsifying a silanol-functional polysiloxane (I) in water in the absence of any basic or acidic catalyst for silanol polycondensation, adding an organofunctional silane of the formula X-A-Si(R)ₙ(OR′)₃₋ₙ(II), where X represents an organic functional group; A represents a divalent organic linkage; each R represents a hydrocarbyl or substituted hydrocarbyl radical; each R′ represents hydrogen or an alkyl or acyl group; and n=0, 1 or 2, to the aqueous phase of the resulting emulsion and reacting the —OR′ groups of (II) with the silanol groups of the polysiloxane (I) to form the organo-functional polysiloxane.

2. A process according to claim 1, wherein the silanol-functional polysiloxane (I) is emulsified in the presence of a non-ionic surfactant.

3. A process according to claim 1 or claim 2, wherein the silanol-functional polysiloxane (I), at least one surfactant and water are continuously fed to a high shear mixer in such proportions as to form a viscous oil in water emulsion which is continuously withdrawn from the mixer and is diluted before addition of the organofunctional silane (II).

4. A process according to any of claims 1 to 3 wherein the organic functional group X of silane (II) is an amino group.

5. A process according to any of claims 1 to 4 wherein each group R′ of silane (II) is a methyl radical.

6. A process according to claim 5 wherein the organofunctional silane is 3-aminopropyl trimethoxy silane.

7. A process according to any of claims 1 to 6 wherein a cationic surfactant is added to the emulsion no later than the addition of the organofunctional silane (II).

8. A process according to any of claims 1 to 7 wherein a base is added to the emulsion to catalyse the reaction of the OR′ groups of (II) with the silanol groups of the polysiloxane (I).

9. A process according to any of claims 1 to 8 wherein the organofunctional silane (II) and the silanol-functional polysiloxane (I) are reacted at a temperature below 40° C.

10. An emulsion of an organo-functional polysiloxane prepared by the process of any of claims 1 to 9, characterised in that the emulsion contains less than 2% by weight cyclic polysiloxane based on the total weight of polysiloxane in the emulsion.
